(19) United States
(12) Patent Application Publication
(10) Pub. No.: US 2018/0057429 A1
(43) Pub. Date: Mar. 1, 2018

(54) SYSTEM AND METHOD FOR PREPARING AROMATIC DERIVATIVE

(71) Applicant: LCY CHEMICAL CORP., Kaohsiung City (TW)

(72) Inventors: Chan-Chia HSU, New Taipei City (TW); Sheng-Yao HSUEH, Kaohsiung City (TW)

(21) Appl. No.: 15/685,408

(22) Filed: Aug. 24, 2017

Related U.S. Application Data
(60) Provisional application No. 62/378,831, filed on Aug. 24, 2016.

(30) Foreign Application Priority Data
Aug. 16, 2017 (TW) 106127767

(51) Int. Cl. C07C 17/14; C07C 47/52; B01D 3/00; G01N 21/31

(52) U.S. Cl. CPC C07C 17/14; C07C 47/52; B01D 3/009; B01J 2219/00711; G01N 2021/3155; B01J 2219/00049; G01N 21/314

(57) ABSTRACT

A system for preparing an aromatic derivative is provided, including: a photo-bromination reaction section for performing a photocatalytic reaction of an aromatic hydrocarbon and a brominating agent to form an aromatic hydrocarbon bromide; a substitution reaction section for performing a substitution reaction of the an aromatic hydrocarbon bromide from the photo-bromination reaction section with an alkali base compound or an alkali carboxylate compound to form an aromatic derivative; and a regeneration unit for reacting an alkali metal bromide formed by the substitution reaction section with an acid to form a hydrobromic acid. The regeneration unit is in fluid communication with the photo-bromination reaction section, such that the hydrobromic acid is recycled to the photo-bromination reaction section. A method for preparing the aromatic derivative is also provided.

### CROSS REFERENCE TO RELATED APPLICATIONS

This Application claims priority of U.S. Provisional Application No. 62/378,831, filed on Aug. 24, 2016 and Taiwan Patent Application No. 106127767, filed on Aug. 16, 2017, the entirety of which is incorporated by reference herein.

### BACKGROUND

### Technical Field

The disclosure relates to a system and a method for preparing an aromatic derivative.

### Description of the Related Art

The direct oxidation of a benzyl group of aromatic hydrocarbons is an important chemical reaction to provide the corresponding aromatic derivatives. However, the metal reagent and by-product produced by the above reaction are usually not reusable, resulting in a large amount of contamination and waste, and also incurring a high production cost. For oxidation, the above reaction typically uses halogen elements, especially chlorine, which is relatively cheaper. However, when chlorine is reacted into chloride, electrolysis is required to regenerate the chlorine, thus making recycling difficult. Moreover, in the prior art, an independent two-stage process is required to prepare an aromatic derivative, which leads to complicated steps of the process and decreased production efficiency.

### BRIEF SUMMARY

To address the above issues, the disclosure provides a system and a method for preparing aromatic derivatives that can simplify the steps of the process and increase the yield. Furthermore, the waste generated by the reaction can be recycled and reused, thereby reducing the production cost and improving production efficiency.

According to an embodiment, the disclosure provides a system for preparing an aromatic derivative, comprising: a photo-bromination reaction section for performing a photocatalytic reaction of an aromatic hydrocarbon and a brominating agent to form an aromatic hydrocarbon bromide; a substitution reaction section for performing a substitution reaction of the aromatic hydrocarbon bromide from the photo-bromination reaction section with an alkali base compound or an alkali carboxylate compound to form an aromatic derivative; and a regeneration unit for reacting an alkali metal bromide formed by the substitution reaction section with an acid to form a hydrobromic acid. The regeneration unit is in fluid communication with the photo-bromination reaction section, such that the hydrobromic acid is recycled to the photo-bromination reaction section.

According to another embodiment, the disclosure provides a system for preparing an aromatic derivative, comprising: a reaction tank for containing a reaction solution, wherein the reaction solution comprises an aromatic hydrocarbon and a brominating agent; a lighting device for performing a photo-bromination reaction of the aromatic hydrocarbon and the brominating agent from the reaction tank to form a brominated product stream, wherein the brominated product stream comprises a liquid unreacted aromatic hydrocarbon and a solid aromatic hydrocarbon bromide. The system further comprises: a separation unit for separating the solid aromatic hydrocarbon bromide from the liquid unreacted aromatic hydrocarbon, wherein the separation unit is in fluid communication with the reaction tank, such that the liquid unreacted aromatic hydrocarbon is recycled to the reaction tank; and a substitution reactor for performing a substitution reaction of the solid aromatic hydrocarbon bromide from the separation unit with an alkali base compound or an alkali carboxylate compound to form an aromatic derivative.

According to yet another embodiment, the disclosure provides a method for preparing an aromatic derivative, comprising: (a) performing a photo-bromination reaction of an aromatic hydrocarbon and a brominating agent in a first solvent to form an aromatic hydrocarbon bromide; (b) performing a substitution reaction of the aromatic hydrocarbon bromide with an alkali base compound or an alkali carboxylate compound in a second solvent to form an aromatic derivative; and (c) reacting an alkali metal bromide formed by the substitution reaction with an acid to form a hydrobromic acid, and recycling the hydrobromic acid for step (a).

A detailed description is given in the following embodiments with reference to the accompanying drawings.

### BRIEF DESCRIPTION OF THE DRAWINGS

The disclosure can be more fully understood by reading the subsequent detailed description and examples with references made to the accompanying drawings, wherein:

FIG. 1 illustrates a schematic view of a system for preparing an aromatic derivative in accordance with the first embodiment of the disclosure.

FIG. 2 illustrates a schematic view of a system for preparing an aromatic derivative in accordance with the second embodiment of the disclosure.

FIG. 3 illustrates a schematic view of a system for preparing an aromatic derivative in accordance with the third embodiment of the disclosure.

### DETAILED DESCRIPTION

Even if a plurality of technical features is simultaneously disclosed by any embodiments described below, it is not meant that a person using the disclosure has to simultaneously implement all the technical features of any embodiments. That is, as long as the possibility of implementation is not affected, a person with ordinary skill in the art is able to selectively implement a portion of technical feature(s) rather than all of the technical features as desired or design requirements in accordance with the disclosure of the disclosure, thereby increasing the flexibility of the disclosure.

The disclosure provides a system and a method for preparing an aromatic derivative which can efficiently recycle by-products. Since the by-product generated by the reaction can be recycled and reused, it is possible to reduce the energy consumption, reduce the production cost and improve production efficiency. Furthermore, in some embodiments, since an aromatic derivative can be formed from an aromatic hydrocarbon without purifying the intermediate through a purification unit such as a recrystallization unit or a distillation unit, the steps of the process can be simplified.

The term “molar equivalent” as used herein refers to the moles of one compound divided by the moles of another compound which is actually reacted. For example, 1 molar equivalent of p-xylene corresponds to 2.5 molar equivalents of hydrogen peroxide in an aqueous solution of hydrogen peroxide.

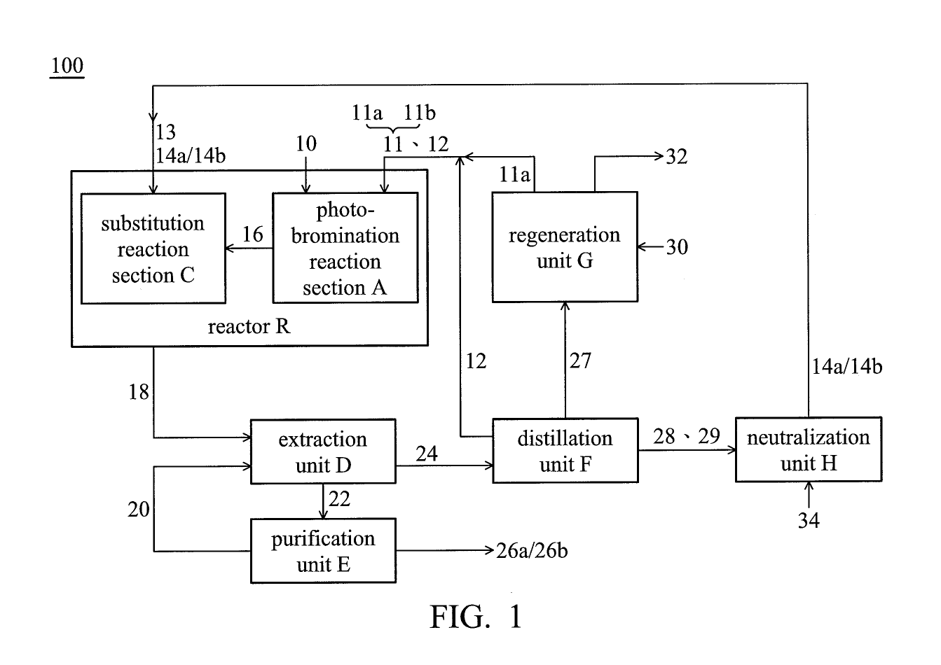

FIG. 1 shows a schematic view of an aromatic derivative preparation system 100 in accordance with the first embodiment. Referring to FIG. 1, the aromatic derivative preparation system 100 includes at least a photo-bromination reaction section A, a substitution reaction section C and a regeneration unit G. In this embodiment, the photo-bromination reaction section A and the substitution reaction section C are located in the same reactor R (e.g., a commercially available batch reactor with a stirring rod). In other embodiments, the photo-bromination reaction section A and the substitution reaction section C may be located in separate reactors.

Referring to FIG. 1, the photo-bromination reaction section A is used to perform a photocatalytic reaction of an aromatic hydrocarbon 10 and a brominating agent 11 to form an aromatic hydrocarbon bromide 16. Furthermore, the substitution reaction section C is used to perform a substitution reaction of the aromatic hydrocarbon bromide 16 from the photo-bromination reaction section A with an alkali base compound 14a or an alkali carboxylate compound 14b to form an aromatic derivative, such as an aromatic alcohol 26a or an aromatic ester 26b in this embodiment. In addition, a regeneration unit G, such as an oxidation-reduction reaction tank, is used to react an alkali metal bromide 27 formed by the substitution reaction section C with an acid (a concentrated sulfuric acid 30 is used in this embodiment, and other acids such as hydrochloric acid may be used in other embodiments based on the design requirement) to form a hydrobromic acid 11a. Furthermore, the regeneration unit G is in fluid communication with the photo-bromination reaction section A, such that the hydrobromic acid 11a is recycled to the photo-bromination reaction section A.

For example, as shown in Formula (1), the photo-bromination reaction section A may be used to perform a photocatalytic reaction of p-xylene and the brominating agent (e.g., a combination of hydrobromic acid (HBr) and aqueous solution of hydrogen peroxide (H₂O₂)) to form α, α′-dibromo-p-xylene. Furthermore, the substitution reaction section C may be used to perform a substitution reaction of α, α′-dibromo-p-xylene from the photo-bromination reaction section A with sodium formate (HCOONa) to form p-xylylene glycol. Moreover, a regeneration unit G may be used to react sodium bromide (NaBr) formed by the substitution reaction section C with sulfuric acid to form hydrobromic acid (HBr), and hydrobromic acid (HBr) is recycled to the photo-bromination reaction section A. It should be noted that the compounds used in Formula (1) are merely examples and are not intended to be limiting.

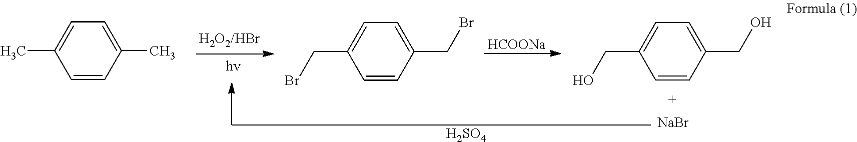

Specifically, the preparation system 100 and the steps of the process of preparing the aromatic derivative will be described in more detail below. As shown in FIG. 1, the preparation system 100 further may further include an extraction unit D, a purification unit E, a distillation unit F and a neutralization unit H.

First, as shown in FIG. 1, the photo-bromination reaction section A is used to perform a photocatalytic reaction of the aromatic hydrocarbon 10. Specifically, the aromatic hydrocarbon 10, the brominating agent 11 (a mixture containing hydrobromic acid (HBr) 11a and aqueous solution of hydrogen peroxide (H₂O₂) 11b is used in this embodiment) and a solvent 12 are added to the photo-bromination reaction section A, and a mixture is obtained after stirring uniformly.

The aromatic hydrocarbon 10 used in the disclosure is not particularly limited. The aromatic hydrocarbon 10 may be, for example, a mono-aromatic cyclic hydrocarbon, a bi-aromatic cyclic hydrocarbon or a tri-aromatic hydrocarbon. In some embodiments, the mono-aromatic cyclic hydrocarbon may be

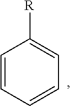

wherein R is a linear or branched alkyl group having 1 to 15 carbon atoms;

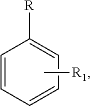

wherein each of R and R₁ is independently a linear or branched alkyl group having 1 to 15 carbon atoms, R₁ may be on any position of the aromatic ring, and the total number of R and R₁ substituent is 6 or less; or a combination thereof. In some embodiments, the bi-aromatic cyclic hydrocarbon may be

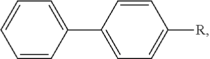

wherein R is a linear or branched alkyl group having 1 to 15 carbon atoms;

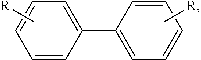

wherein each of R and R₁ is independently a linear or branched alkyl group having 1 to 15 carbon atoms, R and R₁ may be on any position of the aromatic ring, and the total number of R and R₁ substituent is 10 or less;

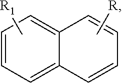

wherein each of R and R₁ is independently a linear or branched alkyl group having 1 to 15 carbon atoms, R and R₁ may be on any position of the aromatic ring, and the total number of R and R₁ substituent is 8 or less;

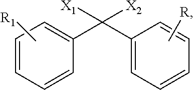

wherein each of R and R₁ is independently a linear or branched alkyl group having 1 to 15 carbon atoms, R and R₁ may be on any position of the aromatic ring, and the total number of R and R₁ substituent is 10 or less; wherein each of X₁ and X₂ is independently hydrogen or a linear, branched or cyclic alkyl group having 1 to 12 carbon atoms;

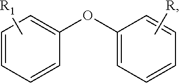

wherein each of R and R₁ is independently a linear or branched alkyl group having 1 to 15 carbon atoms, R and R₁ may be on any position of the aromatic ring, and the total number of R and R₁ substituent is 10 or less;

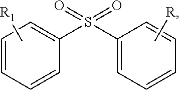

wherein each of R and R₁ is independently a linear or branched alkyl group having 1 to 15 carbon atoms, R and R₁ may be on any position of the aromatic ring, and the total number of R and R₁ substituent is 10 or less; or a combination thereof. In some embodiments, the tri-aromatic cyclic hydrocarbon may be

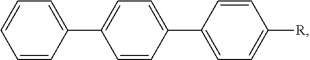

wherein R is a linear or branched alkyl group having 1 to 15 carbon atoms;

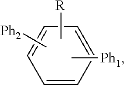

wherein Ph₁ may be

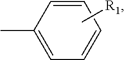

### Ph₂ may be

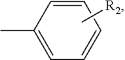

and Ph₁ and Ph₂ may be on any position of the aromatic ring; wherein each of R, R₁ and R₂ is independently a linear or branched alkyl group having 1 to 15 carbon atoms, R, R₁ and R₂ may be on any position of the aromatic ring, and the total number of R, R₁ and R₂ substituent is 14 or less;

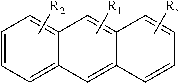

wherein each of R, R₁ and R₂ is independently a linear or branched alkyl group having 1 to 15 carbon atoms, R, R₁ and R₂ may be on any position of the aromatic ring, and the total number of R, R₁ and R₂ substituent is 10 or less;

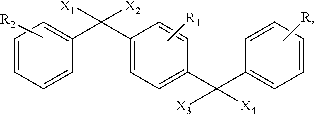

wherein each of R, R₁ and R₂ is independently a linear or branched alkyl group having 1 to 15 carbon atoms, R, R₁ and R₂ may be on any position of the aromatic ring, and the total number of R, R₁ and R₂ substituent is 14 or less; wherein each of X₁, X₂, X₃ and X₄ is independently hydrogen or a linear, branched or cyclic alkyl group having 1 to 12 carbon atoms;

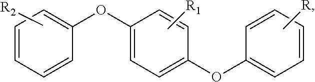

wherein each of R, R₁ and R₂ is independently a linear or branched alkyl group having 1 to 15 carbon atoms, R, R₁ and R₂ may be on any position of the aromatic ring, and the total number of R, R₁ and R₂ substituent is 14 or less;

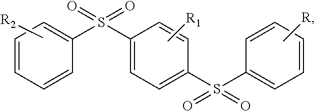

wherein each of R, R₁ and R₂ is independently a linear or branched alkyl group having 1 to 15 carbon atoms, R, R₁ and R₂ may be on any position of the aromatic ring, and the total number of R, R₁ and R₂ substituent is 14 or less; or a combination thereof.

The solvent 12 suitable for the disclosure may be a halogenated hydrocarbon, such as dichloroethane, cyclohexane or other halogenated hydrocarbons which can dissolve the aromatic hydrocarbon.

The brominated agent 11 suitable for the disclosure may be bromine water (Br₂). It should be noted that although bromine water (Br₂) is readily available, it is highly hazardous in handling, delivering and using. Thus, the brominating agent 11 is preferably a mixture having hydrobromic acid (HBr) and aqueous solution of hydrogen peroxide (H₂O₂); a mixture having sodium bromide (NaBr), sulfuric acid (H₂SO₄) and aqueous solution of hydrogen peroxide (H₂O₂); or other mixture capable of producing bromine water (Br₂).

The order of adding the composition of the brominating agent 11 may be adjusted in accordance with the design requirement. For example, after cooling the mixture, the hydrobromic acid 11a is added to the photo-bromination reaction section A. Then, the aqueous solution of hydrogen peroxide 11b is titrated to the above mixture at a temperature of about −10° C. to 30° C. (preferably about 0° C. to 10° C.). In other embodiments, the aqueous solution of hydrogen peroxide 11b may be added before adding the hydrobromic acid 11a. In some embodiments, the molar equivalent ratio of the aromatic hydrocarbon 10, the hydrobromic acid 11a and the solvent 12 is 1:2-3:1-20. In some embodiments, the molar equivalent ratio of the aqueous solution of hydrogen peroxide 11b to the aromatic hydrocarbon 10 is 2-5:1.

Then, the mixture is irradiated with a light source to perform a photocatalytic reaction, thereby forming the aromatic hydrocarbon bromide 16.

In some embodiments, a wavelength of the light source is in a range from about 400 nm to 700 nm, preferably about 420 nm. The power of the light source is in a range from about 20 watt to 200 watt, preferably in a range from about 40 watt to 100 watt, more preferably about 80 watt. In some embodiments, the reaction time of the photo-bromination reaction is in a range from about 0.5 to 24 hours, preferably in a range from about 4 to 12 hours.

It should be noted that since addition of the aqueous solution of hydrogen peroxide 11b to the photo-bromination reaction section A is an exothermic reaction, overheating may produce an unnecessary product. Thus, in the titration process, the temperature of the photo-bromination reaction section A is preferably maintained at about 15° C. or less. If the temperature exceeds 15° C., the addition of the aqueous solution of hydrogen peroxide 11b is stopped until the temperature is lowered.

Referring to FIG. 1, the substitution reaction section C is used to perform the substitution reaction of the aromatic hydrocarbon bromide 16 from the photo-bromination reaction section A. Specifically, water 13 and the alkali base compound 14a or the alkali carboxylate compound 14b are added to the substitution reaction section C, and the substitution reaction of the aromatic hydrocarbon bromide 16 is performed with water 13 and the alkali base compound 14a or the alkali carboxylate compound 14b by refluxing at a temperature of about 80° C. to 160° C. under a pressure of about 1 to 10 atmosphere (atm) (preferably at about 100° C. to 120° C. under about 1 atm, more preferably at about 110° C. to 140° C. under about 1 to 10 atm) for about 0.5 to 24 hours, thereby forming a reaction mixture 18. The reaction mixture 18 includes a crude product of the aromatic alcohol 26a or the aromatic ester 26b, the alkali metal bromide 27, the solvent 12, an acidic compound 28 and water 29.

In some embodiments, the alkali base compound 14a may be sodium hydroxide (NaOH), sodium carbonate (Na₂CO₃), potassium hydroxide (KOH) or a combination thereof. In some embodiments, the alkali carboxylate compound 14b may be sodium formate, sodium acetate or a combination thereof. In some embodiments, the molar equivalent ratio of the alkali base compound 14a or the alkali carboxylate compound 14b to the aromatic hydrocarbon bromide 16 is 2-5:1, preferably 2.5:1.

In some embodiments, a surfactant may be selectively added to the substitution reaction section C to improve the compatibility of the organics and water. In these embodiments, the surfactant may be acetone, 1,4-dioxane or a combination thereof, and the weight ratio of the surfactant to water 13 may be 0.5-4:1.

It should be noted that in this embodiment, the crude product from the photo-bromination reaction section A is directly received by the substitution reaction section C without passing through a purification unit such as a recrystallization unit or a distillation unit. That is, none of steps for purifying (e.g., recrystallizing or distilling) the intermediate is performed during the process of forming the crude product of the aromatic alcohol or the aromatic ester. Accordingly, the aromatic alcohol or the aromatic ester is prepared by an one-pot process, and thus the preparation steps can be simplified.

Still referring to FIG. 1, an extraction unit D (e.g., a commercially available extraction apparatus) is used to separate a product stream (the product stream includes the reaction mixture 18 herein) formed by the substitution reaction section C into an aqueous phase stream and an organic phase stream. The organic phase stream includes the aromatic derivative, such as the aromatic alcohol or the aromatic ester. In other words, an extraction unit D is used to perform an extraction step of the reaction mixture 18 from the substitution reaction section C. Specifically, an extraction solvent 20 is added to the extraction unit D, and the reaction mixture 18 is separated into an organic phase 22 and an aqueous phase 24. The organic phase 22 includes the extraction solvent 20 and the crude product of the aromatic alcohol 26a or the aromatic ester 26b, and the aqueous phase includes the alkali metal bromide 27, the solvent 12, the acidic compound 28 and water 29. In some embodiments, the extraction solvent 20 may be methyl isobutyl ketone (MIBK), ethyl acetate or other suitable solvents. In some embodiments, the weight ratio of the reaction mixture 18 to the extraction solvent 20 is 1:1.

As shown in FIG. 1, a purification unit E is used to perform a purification step of the organic phase 22 from the extraction unit D, such that a product of the aromatic alcohol 26a or the aromatic ester 26b is separated from the extraction solvent 20, and the solvent 20 is recycled to the extraction unit D. In some embodiments, the purification unit E includes a crystallization unit and/or a distillation unit, such as a commercially available crystallization apparatus or distillation apparatus. Thus, the purification step may include a crystallization step, a distillation step, or a combination thereof. For example, the extraction solvent 20 is distilled by a reduced-pressure distillation process and recycled to the extraction unit D. In the embodiments using the system 100, the yield of the aromatic alcohol 26a or the aromatic ester 26b may be up to 62% or more, and in certain examples, the yield may be up to 62%-92%. The purity of the aromatic alcohol 26a or the aromatic ester 26b may be up to 85% or more, and in certain examples, the purity may be up to 90% or more.

Also, the resulting aromatic derivative product, such as the aromatic alcohol 26a or the aromatic ester 26b, is determined by the reactant (such as the alkali base compound 14a or the alkali carboxylate compound 14b) of the substitution reaction. As exemplified by the following Formula (2a), in the case where a reactant of a substitution reaction is an alkali base compound (e.g., sodium hydroxide), a resulting product is an aromatic alcohol. Furthermore, as exemplified by the following Formula (2b), in the case where a reactant of a substitution reaction is an alkali carboxylate compound (e.g., sodium acetate), the resulting product is an aromatic ester. In certain embodiments, in the case where a reactant of a substitution reaction is sodium formate, the resulting product is an aromatic alcohol. For example, as shown in the above-mentioned Formula (1), in the case where a reactant of a substitution reaction is sodium formate, p-xylene eventually forms p-xylylene glycol. It should be noted that the compounds used in Formula (1), Formula (2a) and Formula (2b) are merely examples and are not intended to be limiting.

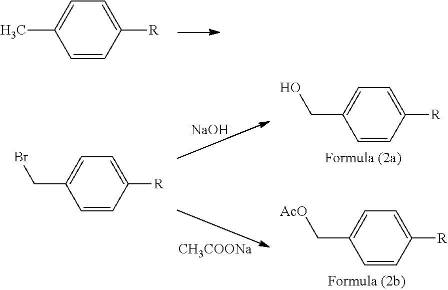

Still referring to FIG. 1, a distillation unit F (e.g., a commercially available distillation apparatus) is used to perform a distillation step of the aqueous phase 24 from the extraction unit D. Specifically, in the distillation unit F, the solvent 12, the acidic compound 28 and water 29 included in the aqueous phase 24 are sequentially distilled off by the difference in boiling point, and the remaining portion is the alkali metal bromide 27. Then, the alkali metal bromide 27, the solvent 12, the acidic compound 28 and water 29 separated by the distillation unit F are recycled respectively, and the details are as follows:

The regeneration unit G is used to react the alkali metal bromide 27 from the distillation unit F to regenerate the hydrobromic acid 11a. Specifically, the concentrated sulfuric acid 30 is added to the regeneration unit G and reacted with the alkali metal bromide 27 to form the hydrobromic acid 11a and an alkali metal salt 32. Furthermore, the regeneration unit G is in fluid communication with the photo-bromination reaction section A, such that the hydrobromic acid 11a obtained by the regeneration unit G is recycled to the photo-bromination reaction section A. In some embodiments, the molar equivalent ratio of the alkali metal bromide 27 to the concentrated sulfuric acid 30 is 1: 2-4. In some embodiments, an additional purification step (e.g., a recrystallization step) of the alkali metal bromide 27 is performed before the alkali metal bromide 27 is moved to the regeneration unit G. It is noted that the reaction of processing the alkali metal bromide 27 to regenerate the hydrobromic acid 11a can be accomplished by simple operations such as heating and distilling under reduced pressure. In contrast, a chlorinating agent (e.g., chlorine gas) used in the prior art requires electrolyzing the chloride produced by the reaction to regenerate and recycle the chlorinating agent. Furthermore, the chlor-alkali industrial process makes enormous demands on the purity of sodium chloride, and thus recycling sodium chloride is relatively costly. Therefore, the system provided by the disclosure can recycle the hydrobromic acid without using the aforementioned electrolysis technology, thereby saving the preparation cost and improving production efficiency.

Furthermore, a neutralization unit H (e.g., a commercially available acid-base neutralization tank) is used to perform a neutralization reaction of the acidic compound 28 and water 29 from the distillation unit F. Specifically, the basic compound 34 is added to the neutralizing unit H, and the neutralization reaction of the acidic compound 28 and water 29 is performed with the basic compound 34 to form the basic alkali metal compound 14a or the alkali metal carboxylate 14b. Furthermore, the neutralization unit H is in fluid communication with the substitution reaction section C, such that the alkali base compound 14a or the alkali carboxylate compound 14b is recycled to the substitution reaction section C.

It will be understood that in this embodiment, a by-product produced by the photo-bromination reaction section A and the substitution reaction section C can be regenerated as a reactant and recycled to the reactor after being processed. Therefore, it is possible to reduce the energy consumption, reduce the production cost and improve production efficiency.

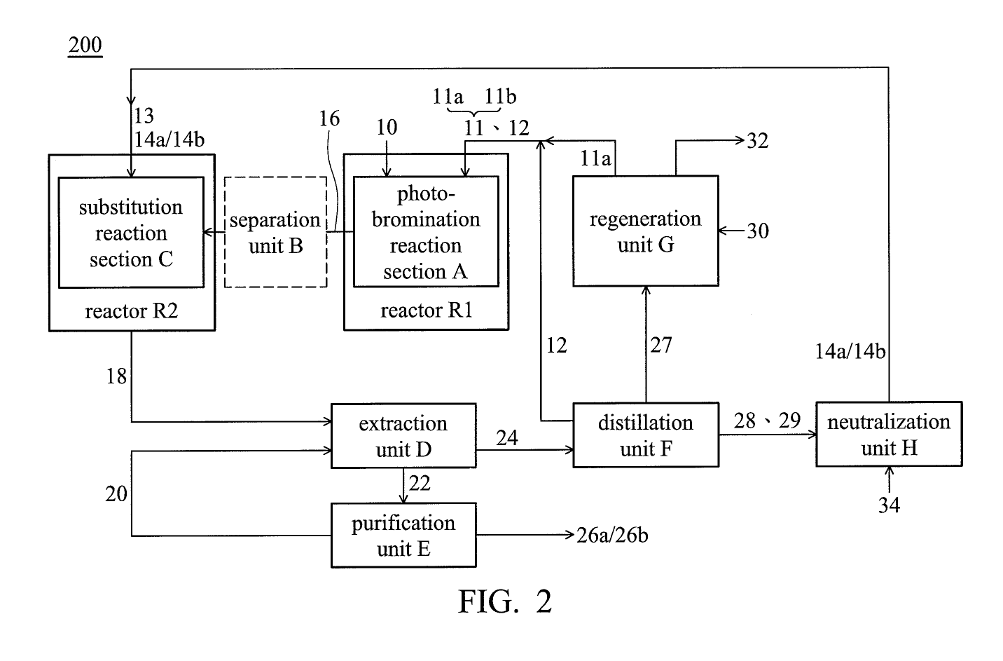

FIG. 2 shows a schematic view of an aromatic derivative preparation system 200 in accordance with the second embodiment. In this embodiment, the photo-bromination reaction and the substitution reaction are performed in separate reactors, thereby improving production efficiency.

Referring to FIG. 2, the preparation system 200 is substantially similar to the preparation system 100 described in the above embodiments. The difference therebetween is that the photo-bromination reaction section A and the substitution reaction section C of the preparation system 200 are located in separate reactors. The reactor can be adjusted according to the design requirement, for example, a continuous reactor can be used.

In detail, after the photocatalytic reaction is performed in the photo-bromination reaction section A of a reactor R1 to form the aromatic hydrocarbon bromide 16, the aromatic hydrocarbon bromide 16 is taken out from the reactor R1 and moved to another reaction R2. Next, the substitution reaction is performed in the substitution reaction section C of the reactor R2 to form a reaction mixture 18. The reaction mixture 18 includes the crude product of the aromatic alcohol 26a or the aromatic ester 26b.

In another embodiment, the preparation system 200 may further include a separation unit B for separating the aromatic hydrocarbon bromide 16 formed by the reactor R1 and then moving the aromatic hydrocarbon bromide 16 to the reactor R2, thereby improving production efficiency.

In the embodiments using the system 200, the yield of the aromatic alcohol 26a or the aromatic ester 26b may be up to 75% or more, and in certain examples, the yield may be up to 75%-96%. The purity of the aromatic alcohol 26a or the aromatic ester 26b may be up to 90% or more, and in certain examples, the purity may be up to 90%-95%.

It should be noted that although the photo-bromination reaction section A and the substitution reaction section C of the preparation system 200 are located in separate reactors, the crude product from the photo-bromination reaction section A is directly received by the substitution reaction section C without passing through a purification unit such as a recrystallization unit or a distillation unit. That is, none of steps for purifying (e.g., recrystallizing or distilling) the intermediate is performed during the process of forming the crude product of the aromatic alcohol or the aromatic ester. Accordingly, the preparation steps can be simplified.

Furthermore, in the preparation system 100 shown in FIG. 1, the photo-bromination reaction and the substitution reaction are performed in the same reactor, so that the raw material is fed into the reactor in batch. In the preparation system 200 shown in FIG. 2, the photo-bromination reaction and the substitution reaction are performed in separate reactors, so that the raw material can be successively fed into the reactor. Thus, compared to the preparation system 100, using the preparation system 200 to prepare the aromatic derivative can possess higher yield and production efficiency.

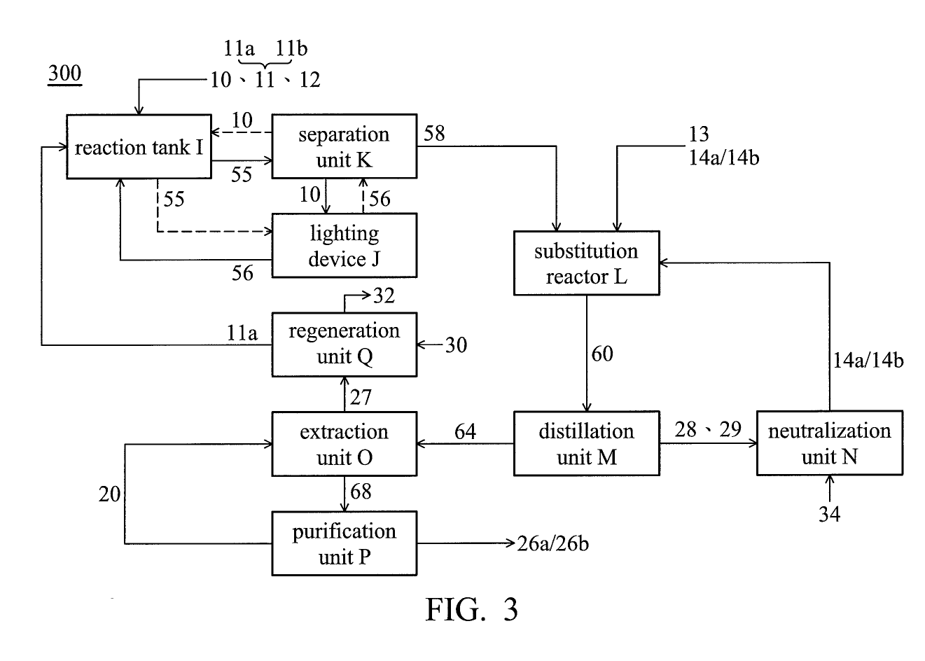

FIG. 3 shows a schematic view of an aromatic derivative preparation system 300 in accordance with the third embodiment. In addition to the advantages of the above embodiments, the present embodiment can further improve production efficiency because the photo-bromination reaction may be continuously reacted in an external circulation system.

Referring to FIG. 3, the aromatic derivative preparation system 300 includes a reaction tank I, a lighting device J, a separation unit K, a substitution reactor L, a distillation unit M, a neutralization unit N, an extraction unit O, a regeneration unit Q and a purification unit P.

As shown in FIG. 3, the reaction tank I (e.g., a commercially available continuous reactor) is used to contain a reaction solution, and the reaction solution includes an aromatic hydrocarbon and a brominating agent. In this embodiment, the reaction solution includes the aromatic hydrocarbon 10, the brominating agent 11 and the solvent 12. After the reaction solution is stirred uniformly, a mixture is obtained. After cooling the mixture, an aqueous solution of hydrogen peroxide is titrated to the mixture to give a reaction mixture 55. The reaction mixture 55 includes a liquid unreacted aromatic hydrocarbon 10 and a solid aromatic bromide 58.

In this embodiment, the aromatic hydrocarbon 10, the brominating agent 11, the solvent 12 and the reaction condition may be the same as those of the first embodiment, which are not described again here in detail.

Still referring to FIG. 3, in the third embodiment, the reaction tank I is in fluid communication with the separation unit K and the lighting device J, and the separation unit K is provided before the lighting device J in the direction of the fluid flow. Furthermore, the reaction mixture 55 from the reaction tank I is sequentially passed through the separation unit K, the lighting device J, and returned to the reaction tank I for a cycle process (presented by the solid line).

In this embodiment, the separation unit K may be a centrifugal device having a filter plate for performing a separation step. Specifically, the reaction mixture 55 obtained from the reaction tank I is moved to the separation unit K. The reaction mixture 55 is then separated into the liquid unreacted aromatic hydrocarbon 10 and the solid aromatic bromide 58, and the solid aromatic bromide 58 is retained in the separation unit K and then moved to the substitution reactor L (to be described later). In other embodiments, the separation unit K may be located in the reaction tank I, allowing the reaction mixture 55 to be separated directly by the separation unit K in the reaction tank I.

Also, the lighting device J has a cooling system and a light source for performing a photo-bromination reaction. In detail, the lighting device J is in fluid communication with the separation unit K, such that the photo-bromination reaction of the liquid unreacted aromatic hydrocarbon 10 from the separation unit K is further performed to form a brominated product stream. The brominated product stream includes the liquid unreacted aromatic hydrocarbon and the solid aromatic bromide.

More specifically, the liquid unreacted aromatic hydrocarbon 10 from the separation unit K is moved to the light device J, and the liquid unreacted aromatic hydrocarbon 10 is irradiated with the light source at a temperature of about −10° C. to 30° C. (preferably about 0° C. to 10° C.) to perform the photo-bromination reaction, thereby forming the brominated product stream (i.e., a brominated mixture 56). The brominated mixture 56 includes the liquid unreacted aromatic hydrocarbon 10 and the solid aromatic bromide 58. In some embodiments, a wavelength of the light source is in a range from about 400 nm to 700 nm, preferably about 420 nm. The power of the light source is in a range from about 20 watt to 200 watt, preferably in a range from about 40 watt to 100 watt, more preferably about 80 watt. In some embodiments, the reaction time of the photo-bromination reaction is in a range from about 0.5 to 24 hours, preferably in a range from about 4 to 12 hours.

Furthermore, the lighting device J is in fluid communication with the reaction tank I, such that the brominated mixture 56 is recycled to the reaction tank I. The liquid unreacted aromatic hydrocarbon 10 and the solid aromatic bromide 58 are moved to the separation unit K once again through the cycle process.

In other embodiments, another separation unit may be additionally provided between the lighting device J and the reaction tank I, and said another separation unit may be in fluid communication with the substitution reactor L. Accordingly, the brominated mixture 56 from the lighting device J is separated, and the liquid unreacted aromatic hydrocarbon 10 is recycled to the reaction tank I while the solid aromatic bromide 58 is retained in said another separation unit and subsequently moved to the substitution reactor L.

Still referring to FIG. 3, in the other embodiment, the reaction tank I is in fluid communication with the lighting device J and the separation unit K, and the separation unit K is provided after the lighting device J in the direction of the fluid flow. Furthermore, the reaction mixture 55 from the reaction tank I is sequentially passed through the lighting device J, the separation unit K, and returned to the reaction tank I for a cycle process (presented by the dotted line).

The lighting device J has a cooling system and a light source for performing a photo-bromination reaction of the aromatic hydrocarbon 10 and the brominating agent 11 of the reaction mixture 55 from the reaction tank I, thereby forming a brominated product stream. The brominated product stream includes the liquid unreacted aromatic hydrocarbon and the solid aromatic bromide. Specifically, the reaction mixture 55 of the reaction tank I is moved to the light device J, and the reaction mixture 55 is irradiated with the light source at a temperature of about −10° C. to 30° C. (preferably about 0° C. to 10° C.) to perform the photo-bromination reaction, thereby forming the brominated product stream (i.e., a brominated mixture 56). The brominated mixture 56 includes the liquid unreacted aromatic hydrocarbon 10 and the solid aromatic bromide 58. In this embodiment, the condition of the photo-bromination reaction may be the same as that of the above embodiment, which is not described again here in detail.

Furthermore, the separation unit K may be a centrifugal device having a filter plate for performing a separation step. Specifically, the brominated mixture 56 obtained from the lighting device J is moved to the separation unit K, and the brominated mixture 56 is then separated into the liquid unreacted aromatic hydrocarbon 10 and the solid aromatic bromide 58. The separation unit K is in fluid communication with the reaction tank I, such that the liquid unreacted aromatic hydrocarbon 10 is recycled to the reaction tank I, while the solid aromatic bromide 58 is retained in the separation unit K and subsequently moved to the substitution reactor L (to be described later).

Since the reaction tank I, the lighting device J and the separation unit K form an external circulation system, the liquid unreacted aromatic hydrocarbon 10 can be repeatedly moved to the lighting device 56 for the photo-bromination reaction until the solid aromatic hydrocarbon bromide 58 is formed. Accordingly, all of the aromatic hydrocarbon reactants can be reacted sufficiently. Furthermore, since most of the solid aromatic bromide 58 is retained in the separation unit K without moving into to the lighting device J through the external circulation system, adverse effects which may reduce the efficiency of the photo-bromination reaction can be prevented. Moreover, the aromatic hydrocarbon 10 and the brominating agent 11 can be continuously added to the reaction tank I, such that the photo-bromination reaction may form a continuous reaction in the external circulation system. Accordingly, the product can be mass produced and the process efficiency can be improved.

Also, in the embodiment in which the separation unit is provided before the lighting device, all the mixture is passed through the separation unit K before moving into the lighting device J. Thus, it is ensured that all the solid substances (e.g., solid aromatic bromide 58) are retained in the separation unit without moving into the lighting device J, thereby preventing adverse effects which may reduce the efficiency of the photo-bromination reaction.

Still referring to FIG. 3, the substitution reaction section L (e.g., a commercially available continuous reactor) is used to perform the substitution reaction of the solid aromatic hydrocarbon bromide 58 from the separation unit K. Specifically, the solid aromatic hydrocarbon bromide 58 from the separation unit K is moved to the substitution reaction section L, and water 53 and the alkali base compound 14a or the alkali carboxylate compound 14b are added to the substitution reaction section L. Furthermore, the substitution reaction is performed by refluxing at a temperature of about 80° C. to 160° C. under a pressure of about 1 to 10 atm (preferably at about 100° C. to 120° C. under about 1 atm, more preferably at about 110° C. to 140° C. under about 1 to 10 atm) for about 0.5 to 24 hours, thereby forming a reaction mixture 60. The reaction mixture 60 includes a crude product of the aromatic alcohol 26a or the aromatic ester 26b, the alkali metal bromide 27, the acidic compound 28 and water 29.

In this embodiment, the alkali base compound 14a, the alkali carboxylate compound 14b, the aromatic hydrocarbon bromide 58 and the reaction condition may be the same as those of the first embodiment, which are not described again here in detail.

The crude product from the separation unit K is directly received by the substitution reaction section L without passing through a purification unit such as a recrystallization unit or a distillation unit. That is, none of steps for purifying (e.g., recrystallizing or distilling) the intermediate is performed during the process of forming the crude product of the aromatic alcohol or the aromatic ester. Accordingly, the preparation steps can be simplified.

As shown in FIG. 3, a distillation unit M (e.g., a commercially available distillation apparatus) is used to perform a distillation step of the reaction mixture 60 from the substitution reaction section L. Specifically, in the distillation unit M, the acidic compound 28 and water 29 included in the reaction mixture 60 are sequentially distilled off by the difference in boiling point, and the remaining portion is a mixture 64. The mixture 64 includes the alkali metal bromide 27 and the crude product of the aromatic alcohol 26a or the aromatic ester 26b.

Furthermore, a neutralization unit N (e.g., a commercially available acid-base neutralization tank) is used to perform a neutralization reaction of the acidic compound 28 and water 29 from the distillation unit M. Specifically, the basic compound 34 is added to the neutralizing unit N, and the neutralization reaction of the acidic compound 28 and water 29 is performed with the basic compound 34 to form the basic alkali metal compound 14a or the alkali metal carboxylate 14b. Furthermore, the neutralization unit N is in fluid communication with the substitution reaction section L, such that the alkali base compound 14a or the alkali carboxylate compound 14b is recycled to the substitution reaction section L.

Still referring to FIG. 3, an extraction unit O is used to perform an extraction step of the mixture 64 from the distillation unit M. Specifically, an extraction solvent 20 is added to the extraction unit O to separate the mixture 64 into an organic phase 68 and an aqueous phase. The organic phase 68 includes the crude product of the aromatic alcohol 26a or the aromatic ester 26b, and the aqueous phase is the alkali metal bromide 27. In some embodiments, the extraction solvent 20 may include methyl isobutyl ketone (MIBK), ethyl acetate or other suitable solvents. In some embodiments, the weight ratio of the mixture 64 to the extraction solvent 20 is 1:1.

As shown in FIG. 3, a regeneration unit Q (e.g., a commercially available an oxidation-reduction reaction tank) is used to react the alkali metal bromide 27 from the extraction unit O to regenerate the hydrobromic acid 11a. Specifically, a concentrated sulfuric acid 30 is added to the regeneration unit Q and reacted with the alkali metal bromide 27 to form the hydrobromic acid 11a and an alkali metal salt 32. Furthermore, the regeneration unit Q is in fluid communication with the reaction tank I, such that the hydrobromic acid 11a obtained by the regeneration unit Q is recycled to the reaction tank I. In some embodiments, the molar equivalent ratio of the alkali metal bromide 27 to the concentrated sulfuric acid 30 is 1:2-4.

As shown in FIG. 3, a purification unit P is used to perform a purification step of the organic phase 68 from the extraction unit O, such that a product of the aromatic alcohol 26a or the aromatic ester 26b is separated from the extraction solvent 20, and the solvent 20 is recycled to the extraction unit O. In some embodiments, the purification unit P includes a crystallization unit and/or a distillation unit. Thus, the purification step may include a crystallization step, a distillation step or a combination thereof. In the embodiments using the preparation system 300, the yield of the aromatic alcohol 26a or the aromatic ester 26b may be up to 78% or more, and in certain examples, the yield may be up to 78%-97%. The purity of the aromatic alcohol 26a or the aromatic ester 26b may be up to 92%, and in certain examples, the purity may be up to 95%.

In this embodiment, since the photo-bromination reaction can be performed continuously, all of the aromatic hydrocarbon reactants can be reacted sufficiently. Therefore, production efficiency can be improved and the yield of the aromatic derivative can be increased.

In summary, the disclosure provides a system and a method for preparing an aromatic derivative. Since the by-product generated by the reaction can be recycled and reused, it is possible to reduce the energy consumption, reduce the production cost and improve production efficiency. Furthermore, in certain embodiments, since an aromatic derivative can be formed from an aromatic hydrocarbon without purifying the intermediate through a purification unit such as a recrystallization unit or a distillation unit, the steps of the process can be simplified.

Examples of the aromatic derivatives obtained by the preparation system and the preparation method of the disclosure are provided below. Specifically, the preparation conditions, the reactant formulation, the resulting yield and purity of the aromatic derivatives described in Examples 1-11 are summarized in Table 1 below. The aromatic derivatives of Examples 1 to 5 are prepared by using the preparation system 100 of the first embodiment. The aromatic derivatives of Examples 6 to 8 are prepared by using the preparation system 200 of the above second embodiment. The aromatic derivatives of Examples 9 to 14 are prepared by using the preparation system 300 of the above third embodiment.

In the following Examples, the yield is defined as: the mole number of the aromatic derivative product/the mole number of the aromatic hydrocarbon reactant X 100%.

In the following Examples, the purity of the product is measured by the GC-FID system (Agilent 7890A Single FID SVOA GC System, available form Pace Analytical Services, LLC) as follows:

0.5 g samples were dissolved with 10 g THF, and the samples were sonicated for 0.5 hr. The samples were then filtered and the filtrate was decanted for GC-FID analysis.

GC conditions: J&W DB-1 (60 m×250 um×0.25 um) capillary column; column temperature hold at 80° C. for 2 min, then rising from 80° C. to 260° C. at 10° C./min, and then hold at 260° C. for 5 min.; injection temperature: 300° C.; injection volume: 0.2 μL; mode of sample injection: split flow, split ratio, 100:1; flow rate of carrier gas: 1.50 mL/min; FID temperature: 300° C.

### [Example 1] Preparation of Aromatic Derivative Using System 100

### Photo-Bromination Reaction

1 molar equivalent of p-xylene (available from ACROS OEGANICS, Sigma-Aldrich), 2.05 molar equivalents of hydrobromic acid (HBr) (48 wt %) and 2.15 molar equivalents of dichloroethane were added in a glass reactor and stirred with a mechanical stirrer at 1 atm and room temperature.

After the temperature of the mixture was cooled to about 5° C. to 10° C. by an ice-water bath, 2.5 molar equivalents of hydrogen peroxide (35 wt %) was slowly added by using a peristaltic pump. Meanwhile, the mixture was irradiated with a light bulb having a wavelength of 400 to 700 nm and a power of 80 W to perform a photocatalytic reaction. In the process of adding hydrogen peroxide aqueous solution, the system temperature was maintained not exceeding 15° C. If the temperature exceeded 15° C., addition of aqueous solution of hydrogen peroxide was stopped until the temperature was lowered. When addition of aqueous solution of hydrogen peroxide was completed, the reaction was terminated when the color of the mixture solution turned from dark yellow to light yellow. The reaction time was about 6 hours and the reaction system was then raised to room temperature.

### Substitution Reaction

2.1 molar equivalents of sodium formate (available from LCY Chemical Corp.) and water in the amount of 10 times of sodium formate by weight were directly added to the above glass reactor, and a substitution reaction was performed by refluxing at about 100° C. and 1 atm. The dichloroethane was removed and recycled by an oil-water separation device disposed in the glass reactor while water was retained in the reaction system.

The temperature of the system was lowered to room temperature after the substitution reaction was performed for 6 hours. The reaction mixture was moved to an extraction tank, and an extraction solvent of methyl isobutyl ketone (MIBK) was added to the extraction tank. The weight ratio of the reaction mixture to methyl isobutyl ketone (MIBK) was 1:1. Then, the organic phase of the extract was taken out and the solvent was recycled by a rotary concentrator. Accordingly, p-xylylene glycol was obtained. The yield of p-xylylene glycol was 62% and the purity was 85% measured by GC-FID.

The aqueous phase of the extract was distilled under reduced pressure, and formic acid and water were distilled off. The acid value of formic acid was detected, and a corresponding amount of sodium hydroxide (NaOH) was added to perform a neutralization reaction. Accordingly, sodium formate was obtained and recycled subsequently.

The solid left by the reduced-pressure distillation step was sodium bromide, and sodium bromide was purified by recrystallization and dried. 1 molar equivalent of sodium bromide was mixed with 15 molar equivalents of water in a glass bottle, and the aqueous sodium bromide solution was heated to about 80° C. Then, 97% of concentrated sulfuric acid was added dropwise to the aqueous sodium bromide solution, and the dropping time was controlled for about 30 minutes. After the addition of sulfuric acid was completed, water was concentrated together with the generated hydrobromic acid (HBr) at 125° C. and 1 atm through a vacuum concentration apparatus (the azeotropic temperature of water and hydrobromic acid at 1 atm is 125° C.). The distillate was collected and the regenerated aqueous hydrobromic acid solution was obtained.

### [Example 2] Preparation of Aromatic Derivative Using System 100

The reaction steps and the reaction conditions of Example 2 were substantially the same as those of Example 1 except that in the substitution reaction of Example 2, the reactant formulation was replaced with 2.2 molar equivalents of sodium acetate and water in the amount of 10 times of sodium acetate by weight. The resulting product was 1,4-benzenedimethanol diacetate, and the yield and purity of 1,4-benzenedimethanol diacetate was 62% and 95%, respectively. The detailed reaction formulation and reaction conditions are listed in Table 1.

### [Example 3] Preparation of Aromatic Derivative Using System 100

The reaction steps and the reaction conditions of Example 3 were substantially the same as those of Example 1 except that in the substitution reaction of Example 3, the reactant formulation was replaced with 3 molar equivalents of sodium hydroxide, water and 1,4-dioxane (in which the weight ratio of 1,4-dioxane to water was 1:1, and the total weight of 1,4-dioxane and water was 10 times of sodium hydroxide), and the reaction time was increased to 12 hours. The resulting product was p-xylylene glycol, and the yield and purity of p-xylylene glycol was 65% and 92%, respectively. The detailed reaction formulation and reaction conditions are listed in Table 1.

### [Example 4] Preparation of Aromatic Derivative Using System 100

The reaction steps and the reaction conditions of Example 4 were substantially the same as those of Example 1 except that in the photo-bromination reaction of Example 4, the reactant formulation was replaced with 5 molar equivalents of toluene and 1 molar equivalent of hydrobromic acid (48 wt %), and the amount of aqueous solution of hydrogen peroxide was changed to be 1.2 molar equivalents.

Furthermore, in the substitution reaction of Example 4, the reactant formulation was replaced with 2 molar equivalents of sodium hydroxide, water and 1,4-dioxane (in which the weight ratio of 1,4-dioxane to water was 1:1, and the total weight of 1,4-dioxane and water was 10 times of sodium hydroxide), and the reaction time was reduced to be 6 hours. The resulting product was benzyl alcohol, and the yield and purity of benzyl alcohol was 83% and 93%, respectively. The detailed reaction formulation and reaction conditions are listed in Table 1.

### [Example 5] Preparation of Aromatic Derivative Using System 100

The reaction steps and the reaction conditions of Example 5 were substantially the same as those of Example 1 except that in the photo-bromination reaction of Example 4, the reactant formulation was replaced with 5 molar equivalents of toluene and 1 molar equivalent of hydrobromic acid (48 wt %), and the amount of aqueous solution of hydrogen peroxide was changed to be 1.2 molar equivalents.

Furthermore, in the substitution reaction of Example 5, the reactant formulation was replaced with 2.2 molar equivalents of sodium methacrylate, 1000 ppm of hydroquinone monomethyl ether (as an inhibitor) and water in the amount of 10 times of sodium methacrylate by weight. The resulting product was benzyl methacrylate, and the yield and purity of benzyl methacrylate was 76% and 90%, respectively. The detailed reaction formulation and reaction conditions are listed in Table 1.

### [Example 6] Preparation of Aromatic Derivative Using System 200

### Photo-Bromination Reaction

1 molar equivalent of p-xylene (available from ACROS OEGANICS, Sigma-Aldrich), 2.05 molar equivalents of hydrobromic acid (HBr) (48 wt %) and 2.15 molar equivalents of dichloroethane were added in a glass reactor and stirred with a mechanical stirrer at 1 atm and room temperature.

After the temperature of the mixture was cooled to about 5° C. to 10° C. by an ice-water bath, 2.5 molar equivalents of hydrogen peroxide (35 wt %) was slowly added by using a peristaltic pump. Meanwhile, the mixture was irradiated with a light bulb having a wavelength of 400 to 700 nm and a power of 80 W to perform a photocatalytic reaction. In the process of adding hydrogen peroxide aqueous solution, the system temperature was maintained not exceeding 15° C. If the temperature exceeded 15° C., addition of aqueous solution of hydrogen peroxide was stopped until the temperature was lowered. When addition of aqueous solution of hydrogen peroxide was completed, the reaction was terminated when the color of the mixture solution turned from dark yellow to light yellow. The reaction time was about 6 hours and the reaction system was then raised to room temperature. A mixture solution having the white solid was obtained in the glass reactor.

### Substitution Reaction

Next, the solid of the mixture solution was separated from the liquid by a Buchner funnel, and a white solid intermediate product (i.e., α,α′-dibromo-p-xylene) was obtained. The white solid intermediate product was weighed and moved to another reactor. Furthermore, 2.1 molar equivalents of sodium formate and water in the amount of 10 times of sodium formate by weight were directly added to the other glass reactor, and a substitution reaction was performed by refluxing at about 100° C. and 1 atm.

The temperature of the system was lowered to room temperature after the substitution reaction was performed for 8 hours. The reaction mixture was moved to an extraction tank, and an extraction solvent of methyl isobutyl ketone (MIBK) was added to the extraction tank. The weight ratio of the reaction mixture to methyl isobutyl ketone (MIBK) was 1:1. Then, the organic phase of the extract was taken out and the solvent was recycled by a rotary concentrator. Accordingly, p-xylylene glycol was obtained. The yield of p-xylylene glycol was 92% and the purity was 90% measured by GC-FID.

The aqueous phase of the extract was distilled under reduced pressure, and formic acid and water were distilled off. The acid value of formic acid was detected, and a corresponding amount of sodium hydroxide (NaOH) was added to perform a neutralization reaction. Accordingly, sodium formate was obtained and recycled subsequently.

The solid left by the reduced-pressure distillation step was sodium bromide, and sodium bromide was purified by recrystallization and dried. 1 molar equivalent of sodium bromide was mixed with 15 molar equivalents of water in a glass bottle, and the aqueous sodium bromide solution was heated to about 80° C. Then, 97% of concentrated sulfuric acid was added dropwise to the aqueous sodium bromide solution, and the dropping time was controlled for about 30 minutes. After the addition of sulfuric acid was completed, completed, water was concentrated together with the generated hydrobromic acid (HBr) at 125° C. and 1 atm through a vacuum concentration apparatus (the azeotropic temperature of water and hydrobromic acid at 1 atm is 125 t). The distillate was collected and the regenerated aqueous hydrobromic acid solution was obtained.

### [Example 7] Preparation of Aromatic Derivative Using System 200

The reaction steps and the reaction conditions of Example 7 were substantially the same as those of Example 6 except that in the substitution reaction of Example 7, the reactant formulation was replaced with 2.2 molar equivalents of sodium acetate and water in the amount of 10 times of sodium acetate by weight. The resulting product was 1,4-benzenedimethanol diacetate, and the yield and purity of 1,4-benzenedimethanol diacetate was 96% and 95%, respectively. The detailed reaction formulation and reaction conditions are listed in Table 1.

### [Example 8] Preparation of Aromatic Derivative Using System 200

The reaction steps and the reaction conditions of Example 8 were substantially the same as those of Example 6 except that in the substitution reaction of Example 8, the reactant formulation was replaced with 3 molar equivalents of sodium hydroxide, water and 1,4-dioxane (in which the weight ratio of 1,4-dioxane to water was 1:1, and the total weight of 1,4-dioxane and water was 10 times of sodium hydroxide), and the reaction time was increased to 12 hours. The resulting product was p-xylylene glycol, and the yield and purity of p-xylylene glycol was 75% and 93%, respectively. The detailed reaction formulation and reaction conditions are listed in Table 1.

### [Example 9] Preparation of Aromatic Derivative Using System 300

### Photo-Bromination Reaction

1 molar equivalent of p-xylene, 2.05 molar equivalents of hydrobromic acid (HBr) (48 wt %) and 2.15 molar equivalents of dichloroethane were added in a glass reactor having a Teflon filter plate and stirred with a mechanical stirrer at 1 atm and room temperature.

After the temperature of the mixture was lowered to about 5° C. to 10° C. by an ice-water bath, 2.5 molar equivalents of hydrogen peroxide (35 wt %) was slowly added to the glass reactor by using a peristaltic pump.

The reaction solution of the glass reactor was pumped into a lighting reactor having a cooling system by using another peristaltic pump. The reaction solution was irradiated with a light bulb having a wavelength of 400 to 700 nm and a power of 80 W to perform a photocatalytic reaction, thereby forming a mixture solution having a solid. The mixture solution was returned to the original glass reactor to form an external circulation system. The temperature of the entire external circulation system was maintained not exceeding 15° C. If the temperature exceeded 15° C., addition of aqueous solution of hydrogen peroxide was stopped until the temperature was lowered.

The solid was retained in the glass reactor by the Teflon filter plate of the glass reactor, so that the solid did not move into to the lighting reactor through the external circulation system. Accordingly, adverse effects which may reduce the efficiency of the lighting reaction could be prevented. When addition of aqueous solution of hydrogen peroxide was completed, the reaction was terminated depending on the color of the mixture solution from dark yellow to light yellow. The reaction time was about 6 hours.

### Substitution Reaction

The solid of the mixture solution was separated from the liquid by a Buchner funnel, and a white solid intermediate product (i.e., α, α′-dibromo-p-xylene) was obtained. The white solid intermediate product was weighed and moved to another reactor. Furthermore, 2.1 molar equivalents of sodium formate and water in the amount of 10 times of sodium formate by weight were directly added to the other glass reactor, and a substitution reaction was performed by refluxing at about 100° C. and 1 atm.

The temperature of the system was lowered to room temperature after the substitution reaction was performed for 8 hours. The reaction mixture was distilled under reduced pressure, and formic acid and water were distilled off. The acid value of formic acid was detected, and a corresponding amount of sodium hydroxide (NaOH) was added to perform a neutralization reaction. Accordingly, sodium formate was obtained and recycled subsequently.

The solid left by the reduced-pressure distillation step was moved to an extraction tank, and an extraction solvent of methyl isobutyl ketone (MIBK) was added to the extraction tank. The weight ratio of the reaction mixture to methyl isobutyl ketone (MIBK) was 1:1. Then, the organic phase of the extract was taken out and the solvent was recycled by a rotary concentrator. Accordingly, p-xylylene glycol was obtained. The yield of p-xylylene glycol was 95% and the purity was 92% measured by GC-FID.

Furthermore, the aqueous phase of the extract was sodium bromide, and sodium bromide was purified by recrystallization and dried. 1 molar equivalent of sodium bromide was mixed with 15 molar equivalents of water in a glass bottle, and the aqueous sodium bromide solution was heated to about 80° C. Then, 97% of concentrated sulfuric acid was added dropwise to the aqueous sodium bromide solution, and the dropping time was controlled for about 30 minutes. After the addition of sulfuric acid was completed, water was concentrated together with the generated hydrobromic acid (HBr) at 125° C. and 1 atm through a vacuum concentration apparatus (the azeotropic temperature of water and hydrobromic acid at 1 atm is 125 t). The distillate was collected and the regenerated aqueous hydrobromic acid solution was obtained.

### [Example 10] Preparation of Aromatic Derivative Using System 300

The reaction steps and the reaction conditions of Example 10 were substantially the same as those of Example 9 except that in the substitution reaction of Example 10, the reactant formulation was replaced with 2.2 molar equivalents of sodium acetate and water in the amount of 10 times of sodium acetate by weight. The resulting product was 1,4-benzenedimethanol diacetate, and the yield and purity of 1,4-benzenedimethanol diacetate was 95% and 95%, respectively. The detailed reaction formulation and reaction conditions are listed in Table 1.

### [Example 11] Preparation of Aromatic Derivative Using System 300

The reaction steps and the reaction conditions of Example 11 were substantially the same as those of Example 9 except that in the substitution reaction of Example 11, the reactant formulation was replaced with 3 molar equivalents of sodium hydroxide, water and 1,4-dioxane (in which the weight ratio of 1,4-dioxane to water was 1:1, and the total weight of 1,4-dioxane and water was 10 times that of sodium hydroxide), and the reaction time was increased to 12 hours. The resulting product was p-xylylene glycol, and the yield and purity of p-xylylene glycol was 78% and 93%, respectively. The detailed reaction foimulation and reaction conditions are listed in Table 1.

### [Example 12] Preparation of Aromatic Derivative Using System 300

The reaction steps and the reactant formulation of Example 12 were substantially the same as those of Example 9 except that the substitution reaction of Example 12 was performed at 3 atm and 130° C. for 3 hours. The resulting product was p-xylylene glycol, and the yield and purity of p-xylylene glycol was 92% and 94%, respectively. The detailed reaction formulation and reaction conditions are listed in Table 1.

### [Example 13] Preparation of Aromatic Derivative Using System 300

The reaction steps of Example 13 were substantially the same as those of Example 9 except that in the substitution reaction of Example 13, the reactant formulation was replaced with 2.1 molar equivalents of sodium formate and water in the amount of 5 times of sodium formate by weight. Furthermore, the substitution reaction of Example 13 was performed at 3 atm and 130° C. for 1 hour. The resulting product was p-xylylene glycol, and the yield and purity of p-xylylene glycol was 93% and 92%, respectively. The detailed reaction formulation and reaction conditions are listed in Table 1.

### [Example 14] Preparation of Aromatic Derivative Using System 300

The reaction steps and the reactant formulation of Example 14 were substantially the same as those of Example 9 except that the substitution reaction of Example 14 was performed at 4 atm and 140° C. for 1 hour. The resulting product was p-xylylene glycol, and the yield and purity of p-xylylene glycol was 97% and 94%, respectively. The detailed reaction formulation and reaction conditions are listed in Table 1.

TABLE 1

| | Photo-bromination reaction | | | | | | Substitution reaction | | | | | | | | |
|---|---|---|---|---|---|---|---|---|---|---|---|---|---|---|---|
| | | Amount | Temperature | Pressure | Wave-length(nm)/power(W) | Time | | | Temperature | Pressure | Time | | | Yield | Purity |
| Example | Reactant | (molar equivalent) | (° C.) | (atm) | of light source | (hr) | Reactant | Amount | (° C.) | (atm) | (hr) | System | Product | (%) | (%) |
| 1 | p-xylene | 1 | 5~10 | 1 | 400~700/80 | 6 | sodium formate | 2.1 molar | 100 | 1 | 8 | 100 | p-xylylene | 62% | 85% |
| | hydrobromic | 2.05 | | | | | | equivalents | | | | | glycol | | |
| | acid | | | | | | | | | | | | | | |
| | aqueous | 2.5 | | | | | water | 10 times of | | | | | | | |
| | solution of | | | | | | | sodium formate | | | | | | | |
| | hydrogen | | | | | | | by weight | | | | | | | |
| | peroxide | | | | | | | | | | | | | | |
| | dichloroethane | 2.15 | | | | | | | | | | | | | |
| 2 | p-xylene | 1 | 5~10 | 1 | 400~700/80 | 6 | sodium | 2.2 molar | 100 | 1 | 8 | 100 | 1,4-benzendi | 62% | 95% |
| | hydrobromic | 2.05 | | | | | acetate | equivalents | | | | | methanol | | |
| | acid | | | | | | | | | | | | diacetate | | |
| | aqueous | 2.5 | | | | | water | 10 times of | | | | | | | |
| | solution of | | | | | | | sodium | | | | | | | |
| | hydrogen | | | | | | | acetate by | | | | | | | |
| | peroxide | | | | | | | weight | | | | | | | |
| | dichloroethane | 2.15 | | | | | | | | | | | | | |
| 3 | p-xylene | 1 | 5~10 | 1 | 400~700/80 | 6 | sodium | 3 molar | 100 | 1 | 12 | 100 | p-xylylene | 65% | 92% |
| | hydrobromic | 2.05 | | | | | hydroxide | equivalents | | | | | glycol | | |
| | acid | | | | | | | | | | | | | | |
| | aqueous | 2.5 | | | | | 1,4-dioxane/water | 10 times of | | | | | | | |
| | solution of | | | | | | (1/1) | sodium hydroxide | | | | | | | |
| | hydrogen | | | | | | | by weight | | | | | | | |
| | peroxide | | | | | | | | | | | | | | |
| | dichloroethane | 2.15 | | | | | | | | | | | | | |
| 4 | toluene | 5 | 5~10 | 1 | 400~700/80 | 6 | sodium | 2 molar | 100 | 1 | 6 | 100 | benzyl | 83% | 93% |
| | hydrobromic | 1 | | | | | hydroxide | equivalents | | | | | alcohol | | |
| | acid | | | | | | | | | | | | | | |
| | aqueous | 1.2 | | | | | 1,4-dioxane/water | 10 times of | | | | | | | |
| | solution of | | | | | | (1/1) | sodium | | | | | | | |
| | hydrogen | | | | | | | hydroxide | | | | | | | |
| | peroxide | | | | | | | by weight | | | | | | | |
| 5 | toluene | 5 | 5~10 | 1 | 400~700/80 | 6 | sodium | 2.2 molar | 95 | 1 | 8 | 100 | benzyl | 76% | 90% |
| | | | | | | | methacrylate | equivalents | | | | | methacrylate | | |
| | hydrobromic | 1 | | | | | water | 10 times of | | | | | | | |
| | acid | | | | | | | sodium | | | | | | | |
| | | | | | | | | methacrylate | | | | | | | |
| | | | | | | | | by weight | | | | | | | |
| | aqueous | 1.2 | | | | | hydroquinone | 1000 ppm | | | | | | | |
| | solution of | | | | | | monomethyl | | | | | | | | |
| | hydrogen | | | | | | ether | | | | | | | | |
| | peroxide | | | | | | | | | | | | | | |
| 6 | p-xylene | 1 | 5~10 | 1 | 400~700/80 | 6 | sodium formate | 2.1 molar | 100 | 1 | 8 | 200 | p-xylylene | 92% | 90% |
| | hydrobromic | 2.05 | | | | | | equivalents | | | | | glycol | | |
| | acid | | | | | | | | | | | | | | |
| | aqueous | 2.5 | | | | | water | 10 times of | | | | | | | |
| | solution of | | | | | | | sodium formate | | | | | | | |
| | hydrogen | | | | | | | by weight | | | | | | | |
| | peroxide | | | | | | | | | | | | | | |
| | dichloroethane | 2.15 | | | | | | | | | | | | | |
| 7 | p-xylene | 1 | 5~10 | 1 | 400~700/80 | 6 | sodium acetate | 2.2 molar | 100 | 1 | 8 | 200 | 1,4- | 96% | 95% |
| | hydrobromic | 2.05 | | | | | | equivalents | | | | | benzendi | | |
| | acid | | | | | | | | | | | | methanol | | |
| | | | | | | | | | | | | | diacetate | | |
| | aqueous | 2.5 | | | | | water | 10 times of | | | | | | | |
| | solution of | | | | | | | sodium acetate | | | | | | | |
| | hydrogen | | | | | | | by weight | | | | | | | |
| | peroxide | | | | | | | | | | | | | | |
| | dichloroethane | 2.15 | | | | | | | | | | | | | |
| 8 | p-xylene | 1 | 5~10 | 1 | 400~700/80 | 6 | sodium | 3 molar | 100 | 1 | 12 | 200 | p-xylylene | 75% | 93% |
| | hydrobromic | 2.05 | | | | | hydroxide | equivalents | | | | | glycol | | |
| | acid | | | | | | | | | | | | | | |
| | aqueous | 2.5 | | | | | 1,4-dioxane/water | 10 times of | | | | | | | |
| | solution of | | | | | | (1/1) | sodium | | | | | | | |
| | hydrogen | | | | | | | hydroxide by | | | | | | | |
| | peroxide | | | | | | | weight | | | | | | | |
| | dichloroethane | 2.15 | | | | | | | | | | | | | |
| 9 | p-xylene | 1 | 5~10 | 1 | 400~700/80 | 6 | sodium formate | 2.1 molar | 100 | 1 | 8 | 300 | p-xylylene | 95% | 92% |
| | hydrobromic | 2.05 | | | | | | equivalents | | | | | glycol | | |
| | acid | | | | | | | | | | | | | | |
| | aqueous | 2.5 | | | | | water | 10 times of | | | | | | | |
| | solution of | | | | | | | sodium formate | | | | | | | |
| | hydrogen | | | | | | | by weight | | | | | | | |
| | peroxide | | | | | | | | | | | | | | |
| | dichloroethane | 2.15 | | | | | | | | | | | | | |
| 10 | p-xylene | 1 | 5~10 | 1 | 400~700/80 | 6 | sodium acetate | 2.2 molar | 100 | 1 | 8 | 300 | 1,4- | 95% | 95% |
| | hydrobromic | 2.05 | | | | | | equivalents | | | | | benzendi- | | |
| | acid | | | | | | | | | | | | methanol | | |
| | | | | | | | | | | | | | diacetate | | |
| | aqueous | 2.5 | | | | | water | 10 times of | | | | | | | |
| | solution of | | | | | | | sodium acetate | | | | | | | |
| | hydrogen | | | | | | | by weight | | | | | | | |
| | peroxide | | | | | | | | | | | | | | |
| | dichloroethane | 2.15 | | | | | | | | | | | | | |
| 11 | p-xylene | 1 | 5~10 | 1 | 400~700/80 | 6 | sodium hydroxide | 3 molar | 100 | 1 | 12 | 300 | p-xylylene | 78% | 93% |
| | hydrobromic | 2.05 | | | | | | equivalents | | | | | glycol | | |
| | acid | | | | | | | | | | | | | | |
| | aqueous | 2.5 | | | | | 1,4-dioxane/water | 10 times of | | | | | | | |
| | solution of | | | | | | (1/1) | sodium hydroxide | | | | | | | |
| | hydrogen | | | | | | | by weight | | | | | | | |
| | peroxide | | | | | | | | | | | | | | |
| | dichloroethane | 2.15 | | | | | | | | | | | | | |
| 12 | p-xylene | 1 | 5~10 | 1 | 400~700/80 | 6 | sodium formate | 2.1 molar | 130 | 3 | 3 | 300 | p-xylylene | 92% | 94% |
| | hydrobromic | 2.05 | | | | | | equivalents | | | | | glycol | | |
| | acid | | | | | | | | | | | | | | |
| | aqueous | 2.5 | | | | | water | 10 times of | | | | | | | |
| | solution of | | | | | | | sodium formate | | | | | | | |
| | hydrogen | | | | | | | by weight | | | | | | | |
| | peroxide | | | | | | | | | | | | | | |
| | dichloroethane | 2.15 | | | | | | | | | | | | | |
| 13 | p-xylene | 1 | 5~10 | 1 | 400~700/80 | 6 | sodium formate | 2.1 molar | 130 | 3 | 1 | 300 | p-xylylene | 93% | 92% |
| | hydrobromic | 2.05 | | | | | | equivalents | | | | | glycol | | |
| | acid | | | | | | | | | | | | | | |
| | aqueous | 2.5 | | | | | water | 5 times of | | | | | | | |
| | solution of | | | | | | | sodium formate | | | | | | | |
| | hydrogen | | | | | | | by weight | | | | | | | |
| | peroxide | | | | | | | | | | | | | | |
| | dichloroethane | 2.15 | | | | | | | | | | | | | |
| 14 | p-xylene | 1 | 5~10 | 1 | 400~700/80 | 6 | sodium formate | 2.1 molar | 140 | 4 | 1 | 300 | p-xylylene | 97% | 94% |
| | hydrobromic | 2.05 | | | | | | equivalents | | | | | glycol | | |
| | acid | | | | | | | | | | | | | | |
| | aqueous | 2.5 | | | | | water | 10 times of | | | | | | | |
| | solution of | | | | | | | sodium formate | | | | | | | |
| | hydrogen | | | | | | | by weight | | | | | | | |
| | peroxide | | | | | | | | | | | | | | |
| | dichloroethane | 2.15 | | | | | | | | | | | | | |

As shown in Table 1, where the reactant formulation and the reaction conditions were the same (e.g., Example 1, Example 6 and Example 9), Example 6 which used preparation system 200 had a higher yield and purity than Example 1 using preparation system 100. This is because in preparation system 200, the photo-bromination reaction and the substitution reaction were performed in separate reactors. In addition, compared to Example 6 which used preparation system 200, Example 9 using preparation system 300 had a higher yield and purity. This is because the photo-bromination reaction in preparation system 300 was performed continuously to allow sufficient reaction of all p-xylene. Accordingly, the yield and production efficiency were improved.

While the disclosure has been described by way of example and in terms of the preferred embodiments, it should be understood that the disclosure is not limited to the disclosed embodiments. On the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A system for preparing an aromatic derivative, comprising:
a photo-bromination reaction section for performing a photocatalytic reaction of an aromatic hydrocarbon and a brominating agent to form an aromatic hydrocarbon bromide;
a substitution reaction section for performing a substitution reaction of the aromatic hydrocarbon bromide from the photo-bromination reaction section with an alkali base compound or an alkali carboxylate compound to form an aromatic derivative; and
a regeneration unit for reacting an alkali metal bromide formed by the substitution reaction section with an acid to form a hydrobromic acid, wherein the regeneration unit is in fluid communication with the photo-bromination reaction section, such that the hydrobromic acid is recycled to the photo-bromination reaction section.

2. The system for preparing an aromatic derivative as claimed in claim 1, wherein the photo-bromination reaction section and the substitution reaction section are located in the same reactor.

3. The system for preparing an aromatic derivative as claimed in claim 1, wherein the photo-bromination reaction section and the substitution reaction section are located in separate reactors.

4. The system for preparing an aromatic derivative as claimed in claim 1, wherein a crude product from the photo-bromination reaction section is directly received by the substitution reaction section without passing through a recrystallization purification unit or a distillation purification unit.

5. The system for preparing an aromatic derivative as claimed in claim 1, further comprising:
an extraction unit for separating a product stream formed by the substitution reaction section into an aqueous phase stream and an organic phase stream, wherein the organic phase stream comprises the aromatic derivative; and
a distillation unit for separating the aqueous phase stream from the extraction unit into the alkali metal bromide, an acidic compound and a first solvent;
wherein the distillation unit is in fluid communication with the photo-bromination reaction section, such that the first solvent is recycled to the photo-bromination reaction section.

6. The system for preparing an aromatic derivative as claimed in claim 5, further comprising:
a neutralization unit for reacting the acidic compound from the distillation unit with a basic compound to form the alkali base compound or the alkali carboxylate compound, wherein the neutralization unit is in fluid communication with the substitution reaction section, such that the alkali base compound or the alkali carboxylate compound is recycled to the substitution reaction section.

7. The system for preparing an aromatic derivative as claimed in claim 5, further comprising:
a purification unit in fluid communication with the extraction unit for purifying the organic stream from the extraction unit to obtain a purified aromatic derivative.

8. A system for preparing an aromatic derivative, comprising:
a reaction tank for containing a reaction solution, wherein the reaction solution comprises an aromatic hydrocarbon and a brominating agent;
a lighting device for performing a photo-bromination reaction of the aromatic hydrocarbon and the brominating agent from the reaction tank to form a brominated product stream, wherein the brominated product stream comprises a liquid unreacted aromatic hydrocarbon and a solid aromatic hydrocarbon bromide;
a separation unit for separating the solid aromatic hydrocarbon bromide from the liquid unreacted aromatic hydrocarbon, wherein the separation unit is in fluid communication with the reaction tank, such that the liquid unreacted aromatic hydrocarbon is recycled to the reaction tank; and
a substitution reactor for performing a substitution reaction of the solid aromatic hydrocarbon bromide from the separation unit with an alkali base compound or an alkali carboxylate compound to form an aromatic derivative.

9. The system for preparing an aromatic derivative as claimed in claim 8, wherein the lighting device comprises a light source, and a wavelength of the light source is in a range from 400 nm to 700 nm.

10. The system for preparing an aromatic derivative as claimed in claim 9, wherein the wavelength of the light source is about 420 nm.

11. The system for preparing an aromatic derivative as claimed in claim 8, wherein the separation unit is located in the reaction tank.

12. The system for preparing an aromatic derivative as claimed in claim 8, wherein yield of the aromatic derivative is greater than or equal to 78%.

13. The system for preparing an aromatic derivative as claimed in claim 8, further comprising:
a regeneration unit for reacting an alkali metal bromide formed by the substitution reactor with an acid to form a hydrobromic acid, wherein the regeneration unit is in fluid communication with the reaction tank, such that the hydrobromic acid is recycled to the reaction tank.

14. A method for preparing an aromatic derivative, comprising:
(a) performing a photo-bromination reaction of an aromatic hydrocarbon and a brominating agent in a first solvent to form an aromatic hydrocarbon bromide;
(b) performing a substitution reaction of the aromatic hydrocarbon bromide with an alkali base compound or an alkali carboxylate compound in a second solvent to form an aromatic derivative; and
(c) reacting an alkali metal bromide formed by the substitution reaction with an acid to form a hydrobromic acid, and recycling the hydrobromic acid for step (a).

15. The method for preparing an aromatic derivative as claimed in claim 14, wherein neither a recrystallization purification step nor a distillation purification step is performed between step (a) and step (b).

16. The method for preparing an aromatic derivative as claimed in claim 14, wherein step (a) and step (b) are performed in the same reactor.

17. The method for preparing an aromatic derivative as claimed in claim 14, wherein step (a) and step (b) are performed in separate reactors.

18. The method for preparing an aromatic derivative as claimed in claim 14, wherein in the photo-bromination reaction of step (a), a reaction temperature is in a range from −10° C. to 30° C., a reaction time is in a range from 0.5 to 24 hours, and a wavelength of a light source is in a range from 400 nm to 700 nm.

19. The method for preparing an aromatic derivative as claimed in claim 14, wherein in the substitution reaction of step (b), the reaction temperature is in a range of 80° C. to 160° C., the reaction time is in a range of 0.5 to 24 hours and a reaction pressure is in a range of 1 to 10 atm.

20. The method for preparing an aromatic derivative as claimed in claim 14, wherein in step (a), the brominating agent comprises the hydrobromic acid and an aqueous solution of hydrogen peroxide; the aromatic hydrocarbon, the hydrobromic acid, and the first solvent have a molar equivalent ratio of 1:2-3:1-20; and the molar equivalent ratio of the aromatic hydrocarbon to the aqueous solution of hydrogen peroxide is 1:2-5.

21. The method for preparing an aromatic derivative as claimed in claim 14, wherein in step (b), the molar equivalent ratio of the alkali base compound or the alkali carboxylate compound to the aromatic hydrocarbon bromide is 2-5:1.

22. The method for preparing an aromatic derivative as claimed in claim 14, wherein in step (c), the molar equivalent ratio of the alkali metal bromide to the acid is 1:2-4.

23. The method for preparing an aromatic derivative as claimed in claim 14, wherein in step (a), the first solvent comprises halogenated hydrocarbon; wherein in step (b), the second solvent comprises water.

24. The method for preparing an aromatic derivative as claimed in claim 14, wherein in step (a), the brominating agent comprises: (1) bromine water (Br₂); (2) a combination of hydrobromic acid (HBr) and aqueous solution of hydrogen peroxide (H₂O₂); (3) a combination of sodium bromide (NaBr), sulfuric acid (H₂SO₄) and aqueous solution of hydrogen peroxide (H₂O₂); or a combination thereof.

25. The method for preparing an aromatic derivative as claimed in claim 14, wherein in step (b), the alkali base compound comprises sodium hydroxide (NaOH), sodium carbonate (Na₂CO₃), potassium hydroxide (KOH) or a combination thereof.

26. The method for preparing an aromatic derivative as claimed in claim 14, wherein in step (b), the alkali carboxylate compound comprises sodium formate, sodium acetate or a combination thereof.

27. The method for preparing an aromatic derivative as claimed in claim 14, wherein after step (b), the method further comprises:
(d) performing an extraction step of a product stream formed by the substitution reaction with an extraction solvent to separate the product stream into an aqueous phase stream and an organic phase stream, wherein the organic phase stream comprises the aromatic derivative; and
(e) performing a distillation step of the aqueous phase stream to separate the aqueous phase into the alkali metal bromide, an acidic compound and a first solvent.

28. The method for preparing an aromatic derivative as claimed in claim 27, wherein after step (e), the method further comprises:
(f) reacting the acidic compound with a basic compound to form the alkali base compound or the alkali carboxylate compound, and recycling the alkali base compound or the alkali carboxylate compound for step (b); and
(g) recycling the first solvent for step (a).

29. The method for preparing an aromatic derivative as claimed in claim 27, wherein after step (d), the method further comprises:
(h) performing a purification step of the organic phase stream, and recycling the extraction solvent obtained from the purification step for step (d), wherein the purification step comprises a crystallization step, a distillation step or a combination thereof.

30. The method for preparing an aromatic derivative as claimed in claim 14, wherein the aromatic hydrocarbon comprises a mono-aromatic cyclic hydrocarbon, a bi-aromatic cyclic hydrocarbon or a tri-aromatic cyclic hydrocarbon.

31. The method for preparing an aromatic derivative as claimed in claim 30, wherein the mono-aromatic cyclic hydrocarbon comprises 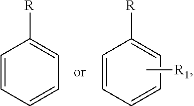
wherein each of R and R₁ is independently a linear or branchedalkyl group having 1 to 15 carbon atoms.
